(19) United States
(12) Patent Application Publication
(10) Pub. No.: US 2022/0211849 A1
(43) Pub. Date: Jul. 7, 2022

(54) SIALYLATED GLYCOPROTEINS

(71) Applicant: Janssen Biotech, Inc., Horsham, PA (US)

(72) Inventor: Siddhesh D. Patil, Cambridge, MA (US)

(21) Appl. No.: 17/602,156

(22) PCT Filed: Apr. 17, 2020
(86) PCT No.: PCT/US20/28863

§ 371 Date: Oct. 7, 2021

Related U.S. Application Data
(60) Provisional application No. 62/836,016, filed on Apr. 18, 2019.

(51) Int. Cl. A61K 39/395; C07K 16/00

(52) U.S. Cl. CPC A61K 39/39591; C07K 16/00; C07K 2317/524; C07K 2317/41; C07K 2317/21

(57) ABSTRACT

Pharmaceutical preparations containing hypersialylated immunoglobulins are described. The preparations are stable to shear stress. The pharmaceutical compositions described herein provide pharmaceutically acceptable hslgG compositions that are stable against shear stress (e.g., a significant a number of subvisible particles do not form when the formulation is subjected to shear stress, such as agitation, for example, durin shippin and thus can be shipped and handled in liquid form.

### CLAIM OF PRIORITY

This application claims the benefit of U.S. Provisional Application Ser. No. 62/836,016, filed on Apr. 18, 2019. The entire contents of the foregoing are incorporated herein by reference.

### BACKGROUND

Intravenous immunoglobulin (IVIg) is prepared from the pooled plasma of human donors (e.g., pooled plasma from at least 1,000 donors) and, while overwhelmingly composed of IgG antibodies, principally IgG1 antibodies, IVIg can also contain trace amount of other antibody subclasses. Commercially available IVIg preparations generally exhibit low levels of sialylation on the Fc domain of the antibodies present. Specifically, the antibodies in commercial IVIg preparations exhibit low levels of disialylation of the branched glycans on the Fc region.

### SUMMARY OF THE INVENTION

Described herein are pharmaceutical compositions comprising hypersialylated immunoglobulins (hslgG). HsIgG has a very high level of sialic acid on the branched glycans on the Fc region of the immunoglobulins, for example, at least 50% (60%, 70%, 80%, 90% or more) of the branched glycans on the Fc region of the immunoglobulins are sialylated via NeuAc-α 2,6-Gal terminal linkages on both the α1,3 arm and the α1,6 arm of the branched glycan.

The pharmaceutical compositions described herein provide pharmaceutically acceptable hslgG compositions that are stable against shear stress (e.g., a significant a number of subvisible particles do not form when the formulation is subjected to shear stress, such as agitation, for example, during shipping) and thus can be shipped and handled in liquid form. The formulations are also stable upon dilution, e.g., dilution in 5% dextrose for intravenous administration. The formulations are stable, for example, at 5° C. for at least 7 months and at 25° C. at least one month, for two years at 2-8° C. and/or two weeks at 15-30° C.

Described herein is a liquid pharmaceutical composition comprising immunoglobulins in 250 mM glycine 0.02% (w/v) polysorbate 20 (pH 4-7), wherein at least 50% of branched glycans on the Fc region of the immunoglobulins are disialylated by way of NeuAc-α 2,6-Gal terminal linkages.

In various embodiments: the concentration of immunoglobulins is 50-250 mg/mL; the concentration of immunoglobulins is 70-130 mg/mL; the concentration of immunoglobulins is 80-120 mg/mL; the concentration of immunoglobulins is 90-110 mg/mL; at least 60%, 70%, 80%, 90% or 95% of branched glycans on the Fc region of the immunoglobulins are disialylated by way of NeuAc-α 2,6-Gal terminal linkages; at least 60%, 70%, 80%, 90% or 95% of branched glycans on the immunoglobulins are disialylated by way of NeuAc-α 2,6-Gal terminal linkages; at least 60%, 70%, 80%, 90% or 95% of branched glycans on the Fab region of the immunoglobulins are disialylated by way of NeuAc-α 2,6-Gal terminal linkages; at least 90% of the immunoglobulins are IgG immunoglobulins; at least 95% of the immunoglobulins are IgG immunoglobulins; 5-20% of the immunoglobulins are dimers; 5-10% of the immunoglobulins are dimers; at least 80% of the immunoglobulins are monomers or dimers; at least 85% of the immunoglobulins are monomers or dimers; at least 90% of the immunoglobulins are monomers or dim5-20% of the IgG immunoglobulins are dimers; 5-10% of the IgG immunoglobulins are dimers; at least 80% of the IgG immunoglobulins are monomers or dimers; at least 85% of the IgG immunoglobulins are monomers or dimers; at least 90% of the IgG immunoglobulins are monomers or dimers; at least 60%, 70%, 80%, 90% or 95% of branched glycans on the Fc region of the IgG immunoglobulins are disialylated by way of NeuAc-α 2,6-Gal terminal linkages; at least 60%, 70%, 80%, 90% or 95% of branched glycans on the IgG immunoglobulins are disialylated by way of NeuAc-α 2,6-Gal terminal linkages; at least 60%, 70%, 80%, 90% or 95% of branched glycans on the Fab region of the IgG immunoglobulins are disialylated by way of NeuAc-α 2,6-Gal terminal linkages; the pH is 4.7-5.5; the pH is 5.1-5.3; the composition has fewer than 1000 particles with a diameter between 10 and 100 micrometers after agitation at 1000 RPM for 8 hours at 2-8° C.; the composition has fewer than 500 particles with a diameter between 10 and 100 micrometers after agitation at 1000 RPM for 8 hours at 2-8° C.; the composition has fewer than 200 particles with a diameter between 10 and 100 micrometers after agitation at 1000 RPM for 8 hours at 2-8° C.; at least 60%, 70%, 80%, 90% or 95% of branched glycans on the Fc region of the immunoglobulins are disialylated by way of NeuAc-α 2,6-Gal terminal linkages after storage for 3, 4, 6, 8, 10, 12, 14, 16, 18, 20, 22 or 24 months at 4° C.; at least 60%, 70%, 80%, 90% or 95% of branched glycans on the immunoglobulins are disialylated by way of NeuAc-α 2,6-Gal terminal linkages after storage for 3, 4, 6, 8, 10, 12, 14, 16, 18, 20, 22 or 24 months at 4° C.; at least 60%, 70%, 80%, 90% or 95% of branched glycans on the Fab region of the immunoglobulins are disialylated by way of NeuAc-α 2,6-Gal terminal linkages after storage for 3, 4, 6, 8, 10, 12, 14, 16, 18, 20, 22 or 24 months at 4° C.; 5-10% of the immunoglobulins are dimers after storage for 3, 4, 6, 8, 10, 12, 14, 16, 18, 20, 22 or 24 months at 4° C.; at least 85% of the immunoglobulins are monomers or dimers after storage for 3, 4, 6, 8, 10, 12, 14, 16, 18, 20, 22 or 24 months at 4° C.; at least 90% of the immunoglobulins are monomers or dimers after storage for 3, 4, 6, 8, 10, 12, 14, 16, 18, 20, 22 or 24 months at 4° C.; the storage is in a sealed US Pharmacopeia Type 1 glass vial; the storage is in a sealed 2R Type 1 glass injection vial.

In hslgG at least 50% (e.g., 60%, 70%, 80%, 82%, 85%, 87%, 90%, 92%, 94%, 95%, 97%, 98% up to and including 100%) of branched glycans on the Fc region of the immunoglobulins have a sialic acid residue on both the α 1,3 arm and the α 1,6 arm (i.e., are disialylated by way of NeuAc-α 2,6-Gal terminal linkages. In some embodiments, in addition to the Fc sialylation, at least 50% (e.g., 60%, 70%, 80%, 82%, 85%, 87%, 90%, 92%, 94%, 95%, 97%, 98% or up to and including 100%) of branched glycans on the Fab region are disialylated by way of NeuAc-α 2,6-Gal terminal linkages. In some cases, at least 85%, (87%, 90%, 92%, 94%, 95%, 97%, 98% or up to and including 100%) of total branched glycans (sum of glycans on the Fc and Fab domains) are disialylated by way of NeuAc-α 2,6-Gal terminal linkages. In some embodiments, less than 50% (e.g., less than 40%, 30%, 20%, 15%, 10%, 5%, 4%, 3%, 2%, 1%) of branched glycans on the Fc region are mono-sialylated (e.g., sialylated only on the α 1,3 arm or the α 1,6 arm) by way of a NeuAc-α 2,6-Gal terminal linkage. The immunoglobulins is HsIgG preparations are primarily IgG antibodies (e.g., at least 80%, 85%, 90%, 95% wt/wt of the immunoglobulins are IgG antibodies of various isotypes.

As used herein, the term “Fc region” refers to a dimer of two “Fc polypeptides,” each “Fc polypeptide” including the constant region of an antibody excluding the CH1 domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or the entire flexible hinge N-terminal to these domains.

As used herein, “glycan” is a sugar, which can be monomers or polymers of sugar residues, such as at least three sugars, and can be linear or branched. A “glycan” can include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a polypeptide, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. Glycoproteins can contain O-linked sugar moieties and/or N-linked sugar moieties.

IVIg is a preparation of pooled, polyvalent immunoglobulins, including all four IgG isotypes, extracted from plasma of at least 1,000 human donors. Among the forms of IVIg approved for use in the United States are Gammagard (Baxter Healthcare Corporation), Gammaplex (Bio Products Laboratory), Bivigam (Biotest Pharmaceuticals Corporation), Carimmune NF (CSL Behring AG), Gamunes-C(Grifols Therapeutics, Inc.) Glebogamma DID (Instituto Grifols, SA) and Octagam (Octapharma Pharmazeutika Produktionsges Mbh). IVIg is approved as a plasma protein replacement therapy for immune deficient patients and for other uses. The level of IVIg Fc glycan sialylation varies among IVIg preparations, but is generally less than 20%. The level of disialylation is generally far lower.

As used herein, an “N-glycosylation site of an Fc polypeptide” refers to an amino acid residue within an Fc polypeptide to which a glycan is N-linked. In some embodiments, an Fc region contains a dimer of Fc polypeptides, and the Fc region comprises two N-glycosylation sites, one on each Fc polypeptide.

As used herein “percent (%) of branched glycans” refers to the number of moles of glycan X relative to total moles of glycans present, wherein X represents the glycan of interest.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

“Pharmaceutical preparations” and “pharmaceutical products” can be included in kits containing the preparation or product and instructions for use.

“Pharmaceutical preparations” and “pharmaceutical products” generally refer to compositions in which the final predetermined level of sialylation has been achieved, and which are free of process impurities. To that end, “pharmaceutical preparations” and “pharmaceutical products” are substantially free of ST6Gal sialyltransferase and/or sialic acid donor (e.g., cytidine 5′-monophospho-N-acetyl neuraminic acid) or the byproducts thereof (e.g., cytidine 5′-monophosphate).

“Pharmaceutical preparations” and “pharmaceutical products” are generally substantially free of other components of a cell in which the glycoproteins were produced (e.g., the endoplasmic reticulum or cytoplasmic proteins and RNA), if recombinant.

By “purified” (or “isolated”) refers to a polynucleotide or a polypeptide that is removed or separated from other components present in its natural environment. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acids. An isolated polynucleotide or polypeptide can be at least 60% free, or at least 75% free, or at least 90% free, or at least 95% free from other components present in natural environment of the indicated polynucleotide or polypeptide.

As used herein, the term “sialylated” refers to a glycan having a terminal sialic acid. The term “mono-sialylated” refers to branched glycans having one terminal sialic acid, e.g., on an α1,3 arm or an α1,6 arm. The term “disialylated” refers to a branched glycan having a terminal sialic acid on two arms, e.g., both an α1,3 arm and an α1,6 arm.

### DESCRIPTION OF DRAWINGS

FIG. 1 schematically depicts an examples of a branched glycan. Light circles are Gal; dark circles are Man; triangles are Fuc, diamonds are NANA; squares are GlcNAc.

FIG. 2 Left panel: Schematic representation of enzymatic sialylation reaction to transform pooled immunoglobulins to hslgG. Right panel: IgG Fc glycan profile for the starting IVIg and for hslgG enzymatically prepared from IVIg. Glycan profiles for the different IgG subclasses are derived via glycopeptide mass spectrometry analysis. Peptide sequences used to quantify glycopeptides for different IgG subclasses are: IgG1=EEQYNSTYR, IgG2/3 EEQFNSTFR, IgG3/4 EEQYNSTFR and EEQFNSTYR.

FIG. 3 depicts vials of hslgG in a conventional formulation used for IVIg that have been subjected to shear stress.

FIG. 4 depicts vials of hslgG in a formulation of that present disclosure that have been subjected to shear stress.

### DETAILED DESCRIPTION

Immunoglobulins are glycosylated at conserved positions in the constant regions of their heavy chain. For example, human IgG has a single N-linked glycosylation site at Asn297 of the CH2 domain. Each immunoglobulin type has a distinct variety of N-linked carbohydrate structures in the constant regions. For human IgG, the core oligosaccharide normally consists of GIcNAc₂Man₃GIcNAc, with differing numbers of outer residues. Variation among individual IgG's can occur via attachment of galactose and/or galactose-sialic acid at one or both terminal GlcNAc or via attachment of a third GlcNAc arm (bisecting GlcNAc).

The present disclosure encompasses, in part, pharmaceutical preparations including pooled human immunoglobulins having an Fc region having particular levels of branched glycans that are sialylated on both of the branched glycans in the Fc region (e.g., with a NeuAc-α 2,6-Gal terminal linkage).

Preparations of pooled, polyvalent human immunoglobulins, including IVIg preparations, are highly complex because they are highly heterogeneous in several regards. They include immunoglobulins pooled from many hundreds or more than 1000 individuals. While at least about 90% or 95% of immunoglobulins are IgG isotype (of all subclasses), other isotypes, including IgA and IgM are present. The immunoglobulins in IVIg and preparations of pooled, polyvalent human immunoglobulins vary in both specificity and glycosylation pattern.

Hypersialylation of pooled, polyvalent immunoglobulins alters the glycans which are present on the immunoglobulins. For some glycans, the alteration entails the addition of one of more galactose molecules and the addition of one or more sialic acid molecules. For other glycans, the alteration entails only the addition of one or more sialic acid molecules. Moreover, while essentially all IgG antibodies, the predominant immunoglobulins in preparations of pooled, polyvalent immunoglobulins, have a glycosylation site on each polypeptide forming Fc region, not all IgG antibodies have a glycosylation site on the Fab domain. Altering the glycosylation of an immunoglobulin preparation alters the structure and activity of the individual immunoglobulins in the preparation and, importantly, alters the interactions between individual immunoglobulins as well as the bulk behavior of preparations of the immunoglobulins.

The widely used formulation used for IVIg preparations is wholly unsuitable for pharmaceutical preparations of hypersialylated immunoglobulins (hslgG) for at least the reason that the formulations, when used for hslgG, are not stable to shear stress that occurs in normal shipping of pharmaceutical formulations. When subjected to this type of shear stress, subvisible particles formed in the hslgG formulations. It is known that such subvisible particles in antibody preparations can cause serious adverse events at the site of injection and off target immune responses. Subvisible particles in antibody preparations can also activate the complement system, cause embolisms, and other negative immunogenic reactions. It was found that the addition of non-ionic surfactants rendered the hslgG preparations more stable to shear stress and greatly reduced the formation of subvisible particles.

Naturally derived polypeptides that can be used to prepare hslgG include, for example, immunoglobulins isolated from pooled human serum. HsIgG can also be prepared from IVIg and polypeptides derived from IVIg. HsIgG can be prepared as described in WO2014/179601. Preparation of hslgG is also described in Washburn et al (Proc Natl Acad Sci USA. 2015 Mar 17;112(11):E1297-306). The level of sialylation in a hslgG preparation can be measured on the Fc domain (e.g., the number of branched glycans that are sialylated on an α1,3 arm, an α1,6 arm, or both, of the branched glycans in the Fc domain), or on the overall sialylation (e.g., the number or percentage of branched glycans that are sialylated on an α1,3 arm, an α1,6 arm, or both, of the branched glycans in the preparation of polypeptides whether on the Fc domain or the Fab domain).

Is some cases, the pooled serum used as a source of immunoglobulins for preparing hslgG is isolated from a specific population of individuals, for example, individuals that produce antibodies against one or more virus, such as COVID-19, SARS, parainfluenza, influenza, but do not have an active infection. In some cases, the immunoglobulins are isolated from a population of individuals in which greater than 50%, 55%, 60%, 75% produce antibodies to a selected virus.

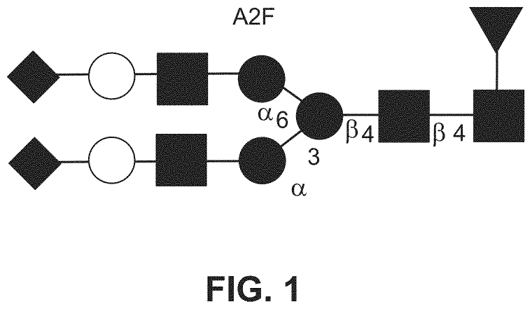

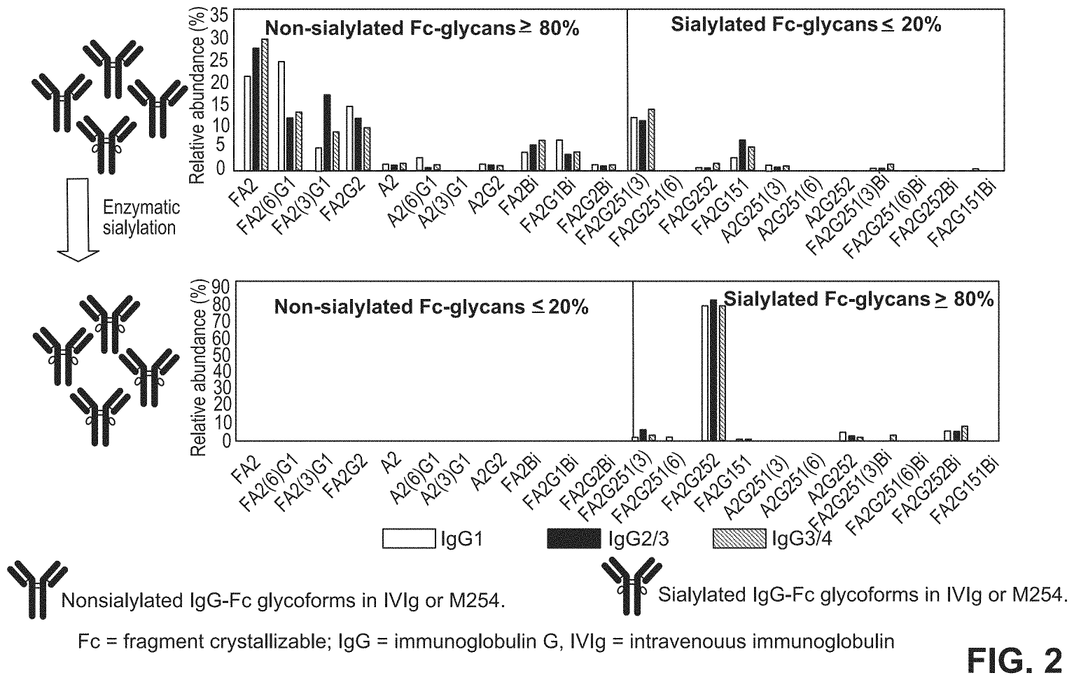

N-linked oligosaccharide chains are added to a protein in the lumen of the endoplasmic reticulum. Specifically, an initial oligosaccharide (typically 14-sugar) is added to the amino group on the side chain of an asparagine residue contained within the target consensus sequence of Asn-X-Ser/Thr, where X may be any amino acid except proline. The structure of this initial oligosaccharide is common to most eukaryotes, and contains three glucose, nine mannose, and two N-acetylglucosamine residues. This initial oligosaccharide chain can be trimmed by specific glycosidase enzymes in the endoplasmic reticulum, resulting in a short, branched core oligosaccharide composed of two N-acetylglucosamine and three mannose residues. One of the branches is referred to in the art as the “α 1,3 arm,” and the second branch is referred to as the “α 1,6 arm,” as shown in FIG. 1.

N-glycans can be subdivided into three distinct groups called “high mannose type,” “hybrid type,” and “complex type,” with a common pentasaccharide core (Man (α 1,6)-(Man(α 1,3))-Man(β1,4)-GlcpNAc(β1,4)-GlcpNAc(β1,N)-Asn) occurring in all three groups.

After initial processing in the endoplasmic reticulum, the polypeptide is transported to the Golgi where further processing may take place. If the glycan is transferred to the Golgi before it is completely trimmed to the core pentasaccharide structure, it results in a “high-mannose glycan.”

Additionally or alternatively, one or more monosaccharides units of N-acetylglucosamine may be added to the core mannose subunits to form a “complex glycan.” Galactose may be added to the N-acetylglucosamine subunits, and sialic acid subunits may be added to the galactose subunits, resulting in chains that terminate with any of a sialic acid, a galactose or an N-acetylglucosamine residue. Additionally, a fucose residue may be added to an N-acetylglucosamine residue of the core oligosaccharide. Each of these additions is catalyzed by specific glycosyl transferases.

“Hybrid glycans” comprise characteristics of both high-mannose and complex glycans. For example, one branch of a hybrid glycan may comprise primarily or exclusively mannose residues, while another branch may comprise N-acetylglucosamine, sialic acid, galactose, and/or fucose sugars.

Sialic acids are a family of 9-carbon monosaccharides with heterocyclic ring structures. They bear a negative charge via a carboxylic acid group attached to the ring as well as other chemical decorations including N-acetyl and N-glycolyl groups. The two main types of sialyl residues found in polypeptides produced in mammalian expression systems are N-acetyl-neuraminic acid (NeuAc) and N-glycolylneuraminic acid (NeuGc). These usually occur as terminal structures attached to galactose (Gal) residues at the non-reducing termini of both N- and O-linked glycans. The glycosidic linkage configurations for these sialyl groups can be either a 2,3 or a 2,6.

Fc regions are glycosylated at conserved, N-linked glycosylation sites. For example, each heavy chain of an IgG antibody has a single N-linked glycosylation site at Asn297 of the C_{H}2 domain. IgA antibodies have N-linked glycosylation sites within the C_{H}2 and C_{H}3 domains, IgE antibodies have N-linked glycosylation sites within the C_{H}3 domain, and IgM antibodies have N-linked glycosylation sites within the C_{H}1, C_{H}2, C_{H}3, and C_{H}4 domains.

Each antibody isotype has a distinct variety of N-linked carbohydrate structures in the constant regions. For example, IgG has a single N-linked biantennary carbohydrate at Asn297 of the C_{H}2 domain in each Fc polypeptide of the Fc region, which also contains the binding sites for C1q and FcγR. For human IgG, the core oligosaccharide normally consists of GIcNAc₂Man₃GIcNAc, with differing numbers of outer residues. Variation among individual IgG can occur via attachment of galactose and/or galactose-sialic acid at one or both terminal GlcNAc or via attachment of a third GlcNAc arm (bisecting GIcNAc).Glycans of polypeptides can be evaluated using any methods known in the art. For example, sialylation of glycan compositions (e.g., level of branched glycans that are sialylated on an α1,3 arm and/or an α1,6 arm) can be characterized using methods described in WO2014/179601.

Composition containing hslgG can include, in addition to antibody monomer, dimers and aggregates of antibodies. In some cases, pH can be used to modulate the percent monomer, dimer, and aggregate in the composition as measured by weight % purity by size exclusion chromatography. In some cases, lowering the pH increases the weight % of monomer+ dimer in the solution. In some cases, lowering the pH increases the weight % monomer in the solution. In some cases, increasing the pH lowers the % monomer in the solution. In some cases, the weight % aggregate is less than or equal to 3.0% wt/wt (e.g., less than or equal to 2.7, 2.5, 2.3, 2.0, 1.7, 1.5, 1.3, 1.0, 0.9, 0.8, 0.7, 0.6, 0.5, 0.4, 0.3, 0.2, or 0.1% wt/wt). In some cases, the weight % Monomer+ Dimer is greater than or equal to 97.0% wt/wt (e.g., greater than or equal to 98% wt/wt or 99% wt/wt). In some cases, the weight % monomer is greater than or equal to 80% wt/wt, 83% wt/wt, 85% wt/wt or 87% wt/wt. In some cases, the pH is less than or equal to 5.3 (e.g., less than or equal to 5.2, 5.1, 5.0, 4.9, 4.8, 4.7, 4.6, 4.5, 4.4, 4.3, 4.2, 4.1, or 4.0). In some cases, the pH of the pharmaceutical compositions is modulated such that the weight % of monomer is altered. In some cases the pH of the pharmaceutical compositions is lowered such that the weight % monomer is increased. In some cases, pH of the pharmaceutical compositions is increased such that the weight % dimer is increased. In some cases, the pH is such that the monomer weight % is greater than or equal to 90% wt/wt (e.g., greater than or equal to 91, 92, 93, 94, 95, 96, 97, 98, or 99% wt/wt).

The invention is further described in the following examples, which do not limit the scope of the invention described in the claims.

### Example 1: Hypersialylated IgG Formulated in 250 mM Glycine

Hypersialylated IgG in which more than 60% of the branched Fc region glycans are disialylated was prepared as generally described in WO2014/179601.

Briefly, IVIg is exposed to a one-pot sequential enzymatic reaction using β1,4 galactosyltransferase 1 (B4-GaIT) and α2,6-sialyltransferase (ST6-Gall) enzymes. The galactosyltransferase enzyme selectively adds galactose residues to pre-existing asparagine-linked glycans in IVIg. The resulting galactosylated glycans serve as substrates to the sialic acid transferase enzyme which selectively adds sialic acid residues to cap the asparagine-linked glycan structures attached to IVIg. Thus, the overall sialylation reaction employed two sugar nucleotides (UDPGal and CMP-NANA. The latter was replenished periodically to increase di-sialylated product relative to monosialylated product. The reaction includes the co-factor manganese chloride.

A representative example of the corresponding IgG-Fc glycan profile for the starting IVIg and the reaction product is shown in the right panel of FIG. 1. The glycan data is shown per IgG subclass. Glycans from IgG3 and IgG4 subclasses cannot be quantified separately. As shown, for IVIg the sum of all the nonsialylated glycans is more than 80% and the sum of all sialylated glycans is <20%. For the reaction product, the sum for all nonsialylated glycans is <20% and the sum for all sialylated glycans is more than 80%. Nomenclature for different glycans listed in the glycoprofile use the Oxford notation for N linked glycans.

IVIg that is not hypersialylated, including commercially available IVIg, is generally stable in glycine and generally does not form subvisible particles when agitated, for example, during shipping. Thus, an initial hypersialylated IgG (hsIVIg) formulation was prepared at 109 mg/mL in 250 mM glycine. The pH was 4.7-5.5. The formulation was clear to opalescent, colorless to pale yellow solution. This formulation was also examined after filtration through a 0.2 micron PES filter membrane into PETG container. Table 1 provides the characteristics of this hypersialylated IgG formulation both pre- and post-filtration. The glycine only formulation appeared to have acceptable product characteristics both pre- and post-filtration.

TABLE 1
Characteristics of hsIgG in 250 mM glycine (pH of 4.7-5.5)

| Attribute | | Units | Unfiltered | Filtered |
|---|---|---|---|---|
| Visual Appearance | | N/A | Opalescent >10 | Opalescent >10 |
| | | | particles | particles |
| Protein concentration | | mg/mL | 109 | 109 |
| Purity by | Monomer | % | 87.36 | 87.16 |
| SEC-HPLC | Aggregates | | 0.87 | 0.73 |
| | Dimer | | 11.72 | 12.01 |
| | Low | | 0.06 | 0.10 |
| | Molecular | | | |
| | Weight | | | |
| | Species | | | |
| Sub-visible | ≥5 μm | Particles/ | 5723 | 17135 |
| Particles | ≥10 μm | ml | 2267 | 5806 |
| | ≥25 μm | | 326 | 379 |
| | ≥50 μm | | 27 | 27 |
| | ≥100 μm | | 0 | 0 |

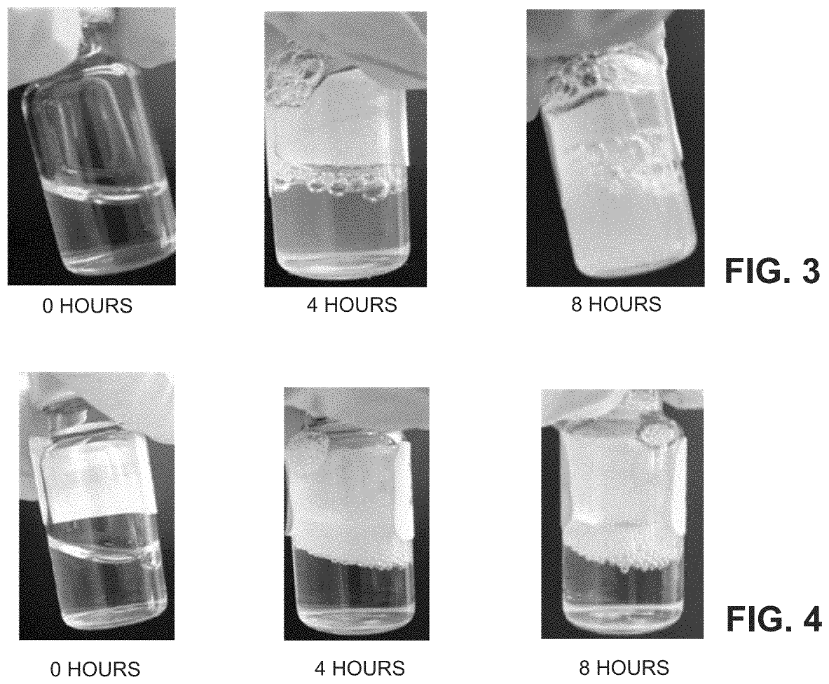

Further study of the in 250 mM glycine (pH of 4.7-5.5) formulation revealed that the hslgG was subject to agitation stress. Samples post-filtration were transferred to glass vials which were shaken at 1000 RPM up to 8 hours under refrigerated temperature (2-8° C.) conditions. Samples were tested before agitation and after 4 and 8 hours post agitation. As can be seen from the photographs in FIG. 3, agitation resulted in the formation of subvisible particles that rendered the solution cloudy. It was found that the 250 mM (pH of 4.7-5.5) formulation became cloudy even when transported by airplane in the passenger compartment, suggesting that the formulation would form subvisible particles during distribution to healthcare providers and patients. Subvisible particles can cause serious adverse events at the site of injection and off target immune responses. The subvisible particles can also activate the complement system, cause embolisms, and other negative immunogenic reactions. Thus, it is important to design a formulation that is stable and will not form subvisible particles upon agitation.

### Example 2: Stabilized Formulation of hslgG

A study was undertaken to develop a formulation of hsIVIG that is stable to agitation stress, but still preserved desirable product attributes present in the 250 mM glycine formulation.

It was found that a low concentration of polysorbate 20 (2-[2-[3,4-bis(2-hydroxyethoxy)oxolan-2-yl]-2-(2-hydroxyethoxy)ethoxy]ethyl dodecanoate; polyoxyethylene (20) sorbitan monolaurate) improved the ability of the hslgG formulation to withstand agitation stress, while retaining desirable product characteristics. Notably, the presence of this non-ionic surfactant did not meaningfully alter the relative amounts of antibody monomers, dimers and higher aggregates.

TABLE 2
Characteristics of hsIgG in 250 mM glycine/0.02%
polysorbate 20 (pH 4.7-5.5)

| Attribute | | Units | Unfiltered | Filtered |
|---|---|---|---|---|
| Visual Appearance | | N/A | Clear | Clear |
| | | | no particles | no particles |
| Protein concentration | | mg/mL | 108 | 106 |
| Purity by | Monomer | % | 87.47 | 86.76 |
| SEC-HPLC | Aggregates | | 0.70 | 0.83 |
| | Dimer | | 11.74 | 12.35 |
| | Low | | 0.09 | 0.05 |
| | Molecular | | | |
| | Weight | | | |
| | Species | | | |
| Sub-visible | ≥5 μm | Particles/ | 9724 | 61 |
| Particles | ≥10 μm | ml | 5030 | 23 |
| | ≥25 μm | | 1266 | 4 |
| | ≥50 μm | | 165 | 0 |
| | ≥100 μm | | 4 | 0 |

Samples of the 250 mM glycine/0.02% polysorbate 20 (pH 4.7-5.5) formulation post-filtration were transferred to glass vials which were shaken at 1000 RPM up to 8 hours under refrigerated temperature (2-8° C.) conditions. Samples were tested before agitation and after 4 and 8 hours post agitation. As can be seen from the photographs in FIG. 4, the formulation was stable to agitation stress.

Table 3 below provides the information on the product attributes of both formulations before and after agitation stress. As can be seen, the addition of 0.02% polysorbate greatly improved agitation stability, but did not alter the monomer/dimer ratio. The addition of 0.02% polysorbate 20 did not significantly protein concentration or monomer, dimer, aggregates and low molecular weight species percentages before or after exposure to agitation stress.

Significantly lower sub-visible particle concentrations were observed in the polysorbate 20-containing formulation compared to the original formulation. Following exposure to agitation stress (4 and 8 hours), comparable particle concentrations were observed compared to their corresponding unstressed condition for both formulations.

TABLE 3
Impact of agitation stress on stability

| | | | | | | 250 mM glycine 20/0.02% | | |
|---|---|---|---|---|---|---|---|---|
| | | | 250 mM glycine 20 (pH 4.7-5.5) | | | polysorbate 20 (pH 4.7-5.5) | | |
| Attribute | | Units | 0 hrs | 4 hrs | 8 hrs | 0 hrs | 4 hrs | 8 hrs |
| Visual Appearance | | N/A | Opalescent >10 | Cloudy; | cloudy | Clear, no | Clear. 3 | Clear, no |
| | | | particles | opalescent, | | particles | particles | particles |
| | | | | many | | | | |
| | | | | particles | | | | |
| Protein concentration | | mg/mL | 109 | 109 | 110 | 106 | 107 | 108 |
| Purity by | Monomer | % | 87.16 | 88.16 | 88.2 | 86.76 | 88.3 | 87.86 |
| SEC-HPLC | Aggregates | | 0.73 | 0.67 | 0.88 | 0.83 | 0.72 | 0.83 |
| | Dimer | | 12.01 | 11.02 | 10.85 | 12.35 | 10.89 | 11.23 |
| | Low | | 0.10 | 0.15 | 0.06 | 0.05 | 0.09 | 0.09 |
| | Molecular | | | | | | | |
| | Weight | | | | | | | |
| | Species | | | | | | | |
| Sub- | ≥5 μm | Particles/ | 17135 | 10736 | 3531 | 61 | 84 | 42 |
| visible | ≥10 μm | ml | 5806 | 4016 | 1369 | 23 | 23 | 8 |
| Particles | ≥25 μm | | 379 | 199 | 191 | 4 | 4 | 0 |
| | ≥50 μm | | 27 | 19 | 23 | 0 | 0 | 0 |
| | ≥100 μm | | 0 | 4 | 4 | 0 | 0 | 0 |

### Example 3: Impact of Dilution in 5% Dextrose Injection

In order to prepare hslgG for administration to patients, concentrated hslgG formulation is sterile filtered through two 0.2 micron PES filter membranes into pre-sterilized USP Type 1 glass vials to create the drug product. The vials are shipped to the clinical sites to be dosed within 72 hours of manufacture (starting from time of filtration). At the clinical sites, the drug product (at 100 mg/mL) is diluted to 60 mg/mL prior to dosing using 5% Dextrose Injection, USP in IV bags. The diluted product is administered to patients using standard infusion lines and systems with an optional 0.2 micron inline filter.

To be successful, the formulation must be stable through these steps, including dilution in 5% Dextrose Injection, USP.

In this study, 100 mg/mL hslgG in 250 mM glycine/0.02% polysorbate 20 was prepared. The resultant solution was filtered through two 0.2 micron PES filter membranes into a PETG container. Sample aliquots transferred were transferred to glass vials and diluted to 60 mg/mL hslgG using 5% Dextrose Injection, USP. Samples were tested at the following conditions:
a) Dilution to 60 mg/mL hslgG in 5% Dextrose Injection, USP;
b) Dilution to 60 mg/mL hslgG in 5% Dextrose Injection, USP+72 hour storage at 5° C.; and
c) Dilution to 60 mg/mL hslgG in 5% Dextrose Injection, USP+72 hour storage at 5° C.+ Filtration using 0.2 micron PES filter.

Under all conditions, the initial formulation, 100 mg/mL hslgG in 250 mM glycine was used as a control. The results of this study are in Table 4, where it can be seen that the 250 mM glycine/0.02% polysorbate 20 formulation exhibited good stability upon dilution.

TABLE 4
Stability upon dilution

| | | | Formulation Type | | | | | |
|---|---|---|---|---|---|---|---|---|
| | | | 100 mg/mL, 250 mM Glycine, | | | 100 mg/mL hsIgG, 250 mM | | |
| | | | pH 4.7-5.5 | | | Glycine, 0.02% PS20, pH 4.7-5.5 | | |
| | | | Dilution to 65 mg/mL in 5% Dextrose Injection | | | | | |
| | | | | | Dilution + | | | Dilution + |
| | | | | | 72 | | | 72 |
| | | | | | hours | | | hours |
| | | | | | storage | | | storage |
| | | | | Dilution + | at 5° C. + | | Dilution + | at 5° C. + |
| | | | | 72 | Filtration | | 72 | Filtration |
| | | | | hour | using 0.2 | | hours | using 0.2 |
| Product Quality Attribute | | | | storage | micron | | storage | micron |
| Attribute | Unit | | Dilution | at 5° C. | PES filter | Dilution | at 5° C. | PES filter |
| Visual Appearance | N/A | | Clear, few | Clear, some | Not | Clear, No | Clear, few | Not |
| | | | particles | particles | tested | particles | particles | tested |
| pH | N/A | | 5.2 | NT | 5.4 | 5.4 | NT | 5.4 |
| Protein | mg/mL | | 65 | NT | 65 | 66 | NT | 64 |
| Concentration | | | | | | | | |
| Sialyation | Mono- | % | NT | NT | 2.38 | NT | NT | 2.20 |
| (% of all | sialylated | | | | | | | |
| glycans | Di- | | NT | NT | 95.88 | NT | NT | 95.91 |
| present) | sialylated | | | | | | | |
| | Total | | NT | NT | 98.26 | NT | NT | 98.11 |
| Purity by | Monomer | % | 0.69 | 0.56 | 0.53 | 0.68 | 0.57 | 0.56 |
| SEC-HPLC | Aggregates | | 11.61 | 11.04 | 10.8 | 11.51 | 10.79 | 10.83 |
| (%) | Dimer | | 87.63 | 88.24 | 88.45 | 87.74 | 88.51 | 88.42 |
| | LMW | | 0.07 | 0.16 | 0.22 | 0.07 | 0.12 | 0.19 |
| Sub-visible | ≥5 μm | Particles | 881 | 2504 | 230 | 310 | 548 | 356 |
| Particles | ≥10 μm | per mL | 368 | 862 | 100 | 119 | 199 | 172 |
| | ≥25 μm | | 65 | 103 | 23 | 15 | 35 | 38 |
| | ≥50 μm | | 12 | 19 | 15 | 8 | 45 | 4 |
| | ≥100 μm | | 0 | 4 | 8 | 0 | 0 | 0 |

In this study, 100 mg/mL (hslgG) in 250 mM glycine/0.02% (w/v) polysorbate 20 was prepared. The resultant solution was filtered through two 0.2 micron PES filter membranes into PETG container. Samples from PETG containers were tested at the following conditions:
a) T zero (stability initiation);
b) 1 month storage at 5° C.;
c) 3 months storage at 5° C. and 25° C.; and
d) 7 months storage at 5° C.

The results of this study are presented in Table 5 where it where it can be seen that the 250 mM glycine/0.02% polysorbate 20 formulation exhibited good stability upon storage.

TABLE 5
Stability upon storage

| | | | Formulation Type | | | | |
|---|---|---|---|---|---|---|---|
| | | | 100 mg/mL hsIgG, 250 mM Glycine, 0.02% PS20 (w/v), pH 4.7-5.5 | | | | |
| | | | Stability Study Time Points | | | | |
| Product Quality Attribute | | | 0 months | 1 month | 3 months | | 7 months |
| Attribute | | Unit | N/A | 25° C. | 5° C. | 25° C. | 5° C. |
| Visual Appearance | | N/A | Clear, no | Clear, no | Clear, no | Clear, no | Clear, no |
| | | | particles | particles | particles | particles | particles |
| Protein Concentration | | mg/mL | 107 | 112 | 109 | 126 | 112 |
| pH | | | 5.43 | 5.39 | 5.47 | 5.26 | 5.4 |
| % | Monomer | % | 2.46 | 3.68 | 2.47 | 2.52 | 3.90 |
| Sialylation | Dimer | | 97.54 | 95.89 | 97.27 | 96.88 | 96.10 |
| | Total | | 100.00 | 99.57 | 99.74 | 99.14 | 99.72 |
| % Purity by | Monomer | % | 86.02 | 86008 | 84.33 | 84.17 | 84.49 |
| SEC-HPLC | Aggregates | | 0.76 | 1.05 | 0.19 | 0.17 | 1.27 |
| | Dimer | | 13.22 | 12.87 | 15.48 | 15.66 | 14.24 |
| Subvisible | ≥5 μm | Particles | 180 | 118 | 1430 | 241 | 2672 |
| particles | ≥10 μm | | 84 | 54 | 421 | 42 | 955 |
| | ≥25 μm | | 8 | 4 | 23 | 4 | 287 |
| | ≥50 μm | | 0 | 0 | 4 | 0 | 102 |

### Example 4: Impact of pH on Purity

In this study three formulations were assessed: H0:100 mg/mL hslgG, 250 mM Glcyine, pH 5.2; H1:100 mg/mL hslgG, 250 mM Glcyine, pH 5.2, 0.02% PS20; H2:100 mg/mL hslgG, 250 mM Glcyine, pH 4.2; and H3:100 mg/mL hslgG, 250 mM Glcyine, pH 4.2, 0.02% PS20. The resultant formulations were filtered through a 0.2 μM PES filter membrane into particulate-free PETG containers. The formulations were then transferred to a 2R Type 1 Glass vial and were tested.

| | | | Protein | | | | |
|---|---|---|---|---|---|---|---|
| | Visual | Clarity | Conc | Purity by SEC-HPLC (%) | | | |
| Sample | Appearance | (NTU) | (mg/mL) | Mono | Agg | Dim | LMV |
| H0 (pH 5.2) | Opalescent, >10 | 31.6 | 109 | 87.36 | 0.87 | 11.72 | 0.06 |
| | particles | | | | | | |
| H1 (pH 5.2, | Clear, no | Not | 108 | 87.47 | 0.7 | 11.74 | 0.09 |
| 0.02% PS20) | particles | tested | | | | | |
| H2 (pH 4.2) | Opalescent, >10 | Not | 108 | 93.64 | 0.4 | 5.86 | 0.1 |
| | particles | tested | | | | | |
| H3 (pH 4.2, | Clear, no | Not | 105 | 93.68 | 0.36 | 5.87 | 0.09 |
| 0.02% PS20) | particles | tested | | | | | |

Samples with lower pH correlated with a higher percent purity by SEC-HPLC of monomer. Samples with lower pH correlated with a higher percent purity by SEC-HPLC of monomer+ dimers and a lower percent aggregates.

### Example 5: Impact of Polysorbate 20 on Sub-Visible Particle Formation

In this study the impact of polysorbate 20 on resistance to shear stress was examined. The following three formulations were prepared: H0:100 mg/mL M254, 250 mM Glcyine, pH 5.2; H1:100 mg/mL M254, 250 mM Glcyine, pH 5.2, 0.02% PS20; H2:100 mg/mL M254, 250 mM Glcyine, pH 4.2; and H3:100 mg/mL M254, 250 mM Glcyine, pH 4.2, 0.02% PS20. The formulations were filtered through a 0.2 μM PES filter membrane into particulate-free PETG containers. The formulations were then transferred to a 2R Type 1 Glass vial and tested.

| | | Protein | MFI (Sub-visible Particles with AR ≤0.7) (#/mL) | | | | |
|---|---|---|---|---|---|---|---|
| | Visual | Conc | ≥5 | ≥10 | ≥25 | ≥50 | ≥100 |
| Sample | Appearance | (mg/mL) | μm | μm | μm | μm | μm |
| H0 (pH 5.2) | Opalescent, >10 | 109 | 17135 | 5806 | 379 | 27 | 0 |
| | particles | | | | | | |
| H1 (pH 5.2, | Clear, no | 106 | 61 | 23 | 4 | 0 | 0 |
| 0.02% PS20) | particles | | | | | | |
| H3 (pH 4.2) | Opalescent, >10 | 106 | 1393 | 486 | 46 | 4 | 0 |
| | particles | | | | | | |
| H3 (pH 4.2, | Clear, no | 104 | 444 | 176 | 27 | 8 | 4 |
| 0.02% PS20) | particles | | | | | | |

### Example 6: Effect of Non-Ionic Surfactants on Shear Stress

The ability of other surfactants to protect 100 mg/ml hsIgG formulations from the adverse effects of shear stress was examined. It was found that PS20 at 0.02%, 0.06% or 0.10% was effective as was polysorbate 80 (2-hydroxyethyl 2-deoxy-3,5-bis-O-(2-hydroxyethyl)-6-O-(2-[(9E)-octadec-9-enoyloxy]ethyl]hexofuranoside; Polyoxyethylene 20 sorbitan monooleate; PS80) or F68 (polyoxyethylene-polyoxypropylene block copolymer CAS Number 9003-11-6. PubChem SID 24898182) at the same concentrations. In each case, vials containing 2.4 ml of the formulation and surfactant-free controls were agitated at 1,000 rpm for four hours at ambient temperature and were analyzed visually, by size exclusion HPLC and particle imaging analysis. Following agitation the non-surfactant samples displayed slight haziness when compared to the static counterparts. The surfactant-containing samples were clear and free of visible particulates. SE_HPLC analysis found that all surfactant-containing samples displayed comparable monomer percentages (88.2%-89.3%). Following agitation the non-surfactant samples displayed higher particle concentrations compared to their static counterparts. Due to their significantly high particle concentrations, the agitated non-surfactant samples could not be digitally filtered. The surfactant containing samples had very low levels of subvisible particles compared to the no surfactant samples.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A liquid pharmaceutical composition comprising immunoglobulins in 250 mM glycine 0.02% (w/v) polysorbate 20 (pH 4-7), wherein at least 50% of branched glycans on the Fc region of the immunoglobulins are disialylated by way of NeuAc-α 2,6-Gal terminal linkages.

2. The liquid pharmaceutical composition of claim 1, wherein the concentration of immunoglobulins is 50-250 mg/mL.

3. (canceled)

4. (canceled)

5. (canceled)

6. The liquid pharmaceutical composition of claim 1, wherein at least 60%, 70%, 80%, 90% or 95% of branched glycans on the Fc domain of the immunoglobulins are disialylated by way of NeuAc-α 2,6-Gal terminal linkages.

7. The liquid pharmaceutical composition of claim 1, wherein at least 60%, 70%, 80%, 90% or 95% of branched glycans on the immunoglobulins are disialylated by way of NeuAc-α 2,6-Gal terminal linkages.

8. The liquid pharmaceutical composition of claim 1, wherein at least 60%, 70%, 80%, 90% or 95% of branched glycans on the Fab domain of the immunoglobulins are disialylated by way of NeuAc-α 2,6-Gal terminal linkages.

9. The liquid pharmaceutical composition of claim 1, wherein at least 90% of the immunoglobulins are IgG immunoglobulins.

10. The liquid pharmaceutical composition of claim 9, wherein at least 95% of the immunoglobulins are IgG immunoglobulins.

11. The liquid pharmaceutical composition of claim 1, wherein 5-20% of the immunoglobulins are dimers.

12. (canceled)

13. The liquid pharmaceutical composition of claim 1, wherein at least 80% of the immunoglobulins are monomers or dimers.

14. (canceled)

15. (canceled)

16. The liquid pharmaceutical composition of claim 1, wherein 5-20% of the IgG immunoglobulins are dimers.

17. The liquid pharmaceutical composition of claim 16, wherein 5-10% of the IgG immunoglobulins are dimers.

18. The liquid pharmaceutical composition of claim 1, wherein at least 80% of the IgG immunoglobulins are monomers or dimers.

19. (canceled)

20. (canceled)

21. The liquid pharmaceutical composition of claim 1, wherein at least 60%, 70%, 80%, 90% or 95% of branched glycans on the Fc domain of the IgG immunoglobulins are disialylated by way of NeuAc-α 2,6-Gal terminal linkages.

22. The liquid pharmaceutical composition of claim 1, wherein at least 60%, 70%, 80%, 90% or 95% of branched glycans on the IgG immunoglobulins are disialylated by way of NeuAc-α 2,6-Gal terminal linkages.

23. The liquid pharmaceutical composition of claim 1, wherein at least 60%, 70%, 80%, 90% or 95% of branched glycans on the Fab domain of the IgG immunoglobulins are disialylated by way of NeuAc-α 2,6-Gal terminal linkages.

24. The liquid pharmaceutical composition of claim 1, wherein the pH is 4.7-5.5.

25. (canceled)

26. The liquid pharmaceutical composition of claim 1, wherein the composition has fewer than 1000 particles with a diameter between 10 and 100 micrometers after agitation at 1000 RPM for 8 hours at 2-8° C.

27. The liquid pharmaceutical composition of claim 1, wherein the composition has fewer than 500 particles with a diameter between 10 and 100 micrometers after agitation at 1000 RPM for 8 hours at 2-8° C.

28. (canceled)

29. The liquid pharmaceutical composition of claim 1, wherein at least 60%, 70%, 80%, 90% or 95% of branched glycans on the Fc domain of the immunoglobulins are disialylated by way of NeuAc-α 2,6-Gal terminal linkages after storage for 3, 4, 6, 8, 10, 12, 14, 16, 18, 20, 22 or 24 months at 4° C.

30. (canceled)

31. (canceled)

32. The liquid pharmaceutical composition of claim 1, wherein 5-10% of the immunoglobulins are dimers after storage for 3, 4, 6, 8, 10, 12, 14, 16, 18, 20, 22 or 24 months at 4° C.

33. The liquid pharmaceutical composition of claim 1, wherein at least 85% of the immunoglobulins are monomers or dimers after storage for 3, 4, 6, 8, 10, 12, 14, 16, 18, 20, 22 or 24 months at 4° C.

34. (canceled)

35. The liquid pharmaceutical composition of claim 1, wherein the formulation is stable at 5° C. for at least 7 months, at 25° C. at least one month, 2-8° C. for two years and/or two weeks at 15-30° C.

36. The liquid pharmaceutical composition of claim 26, wherein the storage is in a sealed US Pharmacopeia Type 1 glass vial.

37. The liquid pharmaceutical composition of claim 26, wherein the storage is in a sealed 2R Type 1 glass injection vial.
